(19) United States
(12) Patent Application Publication
(10) Pub. No.: US 2010/0113443 A1
(43) Pub. Date: May 6, 2010

(54) LIQUID FORMULATIONS OF PHOSPHOLIPASE ENZYME INHIBITORS

(75) Inventors: Mannching Sherry Ku, Thiells, NY (US); Frances Anne Donahue, Garfield, NJ (US); Eugene Lee, Fort Lee, NJ (US)

Correspondence Address: WYETH LLC;PATENT LAW GROUP, 5 GIRALDA FARMS, MADISON, NJ, 07940, US

(73) Assignee: Wyeth, Madison, NJ (US)

(21) Appl. No.: 12/513,071

(22) PCT Filed: Oct. 30, 2007
(86) PCT No.: PCT/US07/82966

§ 371 Date: Dec. 11, 2009

Related U.S. Application Data
(60) Provisional application No. 60/855,663, filed on Oct. 31, 2006.

(51) Int. Cl. A61K 31/404; A61K 31/5377; A61K 31/496; A61K 31/4439; A61K 31/422; A61K 31/4709

(52) U.S. Cl. 514/235.2; 514/415; 514/254.09; 514/339; 514/378; 514/314

(57) ABSTRACT

The present invention relates to liquid formulations of inhibitors of phospholipase enzymes, such as cytosolic PLA₂, compositions containing the same and processes for manufacture thereof.

### CROSS REFERENCE TO RELATED APPLICATIONS

This application claims the benefit of U.S. Provisional Application No. 60/855,663, filed on Oct. 31, 2006, which is incorporated herein by reference in its entirety.

### FIELD OF THE INVENTION

The present invention relates to liquid formulations of inhibitors of phospholipase enzymes, such as cytosolic PLA₂, compositions containing the same and processes for manufacture thereof.

### BACKGROUND OF THE INVENTION

Leukotrienes and prostaglandins are important mediators of inflammation, each of which contributes to the development of an inflammatory response in a different way. Leukotrienes recruit inflammatory cells such as neutrophils to an inflamed site, promote the extravasation of these cells and stimulate release of superoxide and proteases, which damage the tissue. Leukotrienes also play a pathophysiological role in the hypersensitivity experienced by asthmatics {See, e.g. B. Samuelson et al., Science, 237:1171-76 (1987)). Prostaglandins enhance inflammation by increasing blood flow and therefore infiltration of leukocytes to inflamed sites. Prostaglandins also potentiate the pain response induced by stimuli.

Prostaglandins and leukotrienes are unstable and are not stored in cells, but are instead synthesized [W. L. Smith, Biochem. J., 259:315-324 (1989)] from arachidonic acid in response to stimuli. Prostaglandins are produced from arachidonic acid by the action of COX-1 and COX-2 enzymes. Arachidonic acid is also the substrate for the distinct enzyme pathway leading to the production of leukotrienes.

Arachidonic acid, which is fed into these two distinct inflammatory pathways, is released from the sn-2 position of membrane phospholipids by phospholipase A₂ enzymes (hereinafter PLA₂). The reaction catalyzed by PLA₂ is believed to represent the rate-limiting step in the process of lipid mediated biosynthesis and the production of inflammatory prostaglandins and leukotrienes. When the phospholipid substrate of PLA₂ is of the phosphotidyl choline class with an ether linkage in the sn-1 position, the lysophospholipid produced is the immediate precursor of platelet activating factor (hereafter called PAF), another potent mediator of inflammation [S. I. Wasserman, Hospital Practice, 15:49-58 (1988)].

Most anti-inflammatory therapies have focused on preventing production of either prostaglandins or leukotrienes from these distinct pathways, but not on all of them. For example, ibuprofen, aspirin, and indomethacin are all NSAIDs, which inhibit the production of prostaglandins by COX-1/COX-2 inhibition, but have no effect on the inflammatory production of leukotrienes from arachidonic acid in the other pathways. Conversely, zileuton inhibits only the pathway of conversion of arachidonic acid to leukotrienes, without affecting the production of prostaglandins. None of these widely-used anti-inflammatory agents affects the production of PAF.

Consequently the direct inhibition of the activity of PLA₂ has been suggested as a useful mechanism for a therapeutic agent, i.e., to interfere with the inflammatory response. [See, e.g., J. Chang et al, Biochem. Pharmacol., 36:2429-2436 (1987)].

A family of PLA₂ enzymes characterized by the presence of a secretion signal sequenced and ultimately secreted from the cell have been sequenced and structurally defined. These secreted PLA₂s have an approximately 14 kD molecular weight and contain seven disulfide bonds, which are necessary for activity. These PLA₂s are found in large quantities in mammalian pancreas, bee venom, and various snake venoms. [See, e.g., references 13-15 in Chang et al, cited above; and E. A. Dennis, Drug Devel. Res., 10:205-220 (1987).] However, the pancreatic enzyme is believed to serve a digestive function and, as such, should not be important in the production of the inflammatory mediators whose production must be tightly regulated.

The primary structure of the first human non-pancreatic PLA₂ has been determined. This non-pancreatic PLA₂ is found in platelets, synovial fluid, and spleen and is also a secreted enzyme. This enzyme is a member of the aforementioned family. [See J. J. Seilhamer et al., J. Biol. Chem., 264:5335-5338 (1989); R. M. Kramer et al., J. Biol. Chem., 264:5768-5775 (1989); and A. Kando et al., Biochem. Biophys. Res. Comm., 163:42-48 (1989)]. However, it is doubtful that this enzyme is important in the synthesis of prostaglandins, leukotrienes and PAF, since the non-pancreatic PLA₂ is an extracellular protein, which would be difficult to regulate, and the next enzymes in the biosynthetic pathways for these compounds are intracellular proteins. Moreover, there is evidence that PLA₂ is regulated by protein kinase C and G proteins [R. Burch and J. Axelrod, Proc. Natl. Acad. Sci. U.S.A., 84:6374-6378 (1989)], which are cytosolic proteins, which must act on intracellular proteins. It would be impossible for the non-pancreatic PLA₂ to function in the cytosol, since the high reduction potential would reduce the disulfide bonds and inactivate the enzyme.

A murine PLA₂ has been identified in the murine macrophage cell line, designated RAW 264.7. A specific activity of 2 mols/min/mg, resistant to reducing conditions, was reported to be associated with the approximately 60 kD molecule. However, this protein was not purified to homogeneity. [See, C. C. Leslie et al., Biochem. Biophys. Acta., 963:476-492 (1988)]. The references cited above are incorporated by reference herein for information pertaining to the function of the phospholipase enzymes, particularly PLA₂.

A cytosolic phospholipase A₂ alpha (hereinafter “cPLA₂α”) has also been identified and cloned. See, U.S. Pat. Nos. 5,322,776 and 5,354,677, which are incorporated herein in their entirety. The enzyme of these patents is an intracellular PLA₂ enzyme, purified from its natural source or otherwise produced in purified form, which functions intracellularly to produce arachidonic acid in response to inflammatory stimuli.

In addition to the identification of several phospholipase enzymes, efforts have been spent in identifying chemical inhibitors of the action of specific phospholipase enzymes, which inhibitors could be used to treat inflammatory conditions, particularly where inhibition of production of prostaglandins, leukotrienes and PAF are all desired results. Such inhibitors are disclosed, for example, in U.S. Pat. No. 6,797,708 and U.S. patent application Ser. No. 11/442,199 (filed May 26, 2006), each of which is incorporated herein by reference in their entireties.

Given the importance of these compounds as pharmaceutical agents, it can be seen that effective formulations for delivery of the compounds, including those having improved bioavailability, are of great import, and there is an ongoing need for such new formulations.

### SUMMARY OF THE INVENTION

The invention provides pharmaceutical compositions comprising:
a) a pharmaceutically effective amount of an active pharmacological agent having Formula I:

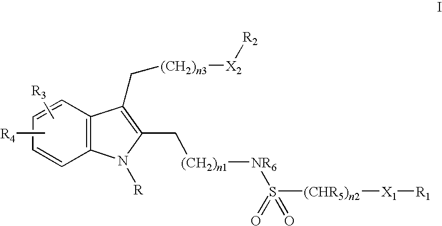

or a pharmaceutically acceptable salt thereof, wherein R, R₁, R₂, R₃, R₄, R₆, X₁, X₂, n₁, n₂, and n₃ are defined as described herein; and
b) a carrier or excipient system comprising a first solubilizer, a second solubilizer, a first diluent, and a second diluent.

The present invention also provides pharmaceutical compositions comprising:
a) a pharmaceutically effective amount of an active pharmacological agent having Formula II:

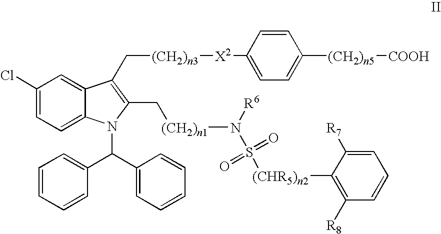

and pharmaceutically acceptable salts thereof, wherein R₅, R₆, R₇, R₈, X², n₁, n₂, n₃, and n₅ are defined as described herein; and
b) a carrier or excipient system comprising a first solubilizer, a second solubilizer, a first diluent and a second diluent.

The invention further provides processes for preparing the pharmaceutical compositions and dosage forms of the invention, and products of the processes.

### BRIEF DESCRIPTION OF THE DRAWINGS

FIG. 1 is a graph depicting the dissolution profile of a formulation according to the invention at different pH.

FIG. 2 is a graph depicting the dissolution profile in simulated fed and fasted state media of a formulation according to the invention.

### DETAILED DESCRIPTION OF THE INVENTION

The present invention provides pharmaceutical compositions and unit dosage forms containing the compositions that have enhanced bioavailability.

In some embodiments, the pharmaceutical composition comprises
a) a pharmaceutically effective amount of an active pharmacological agent having Formula I:

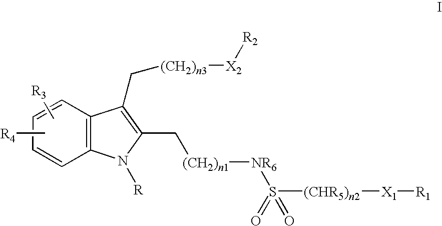

or a pharmaceutically acceptable salt thereof, wherein:
R is selected from the formulae —(CH₂)ₙ-A, —(CH₂)ₙ—S-A, and —(CH₂)ₙ—O-A, wherein A is selected from the moieties:

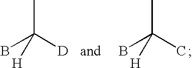

wherein
D is C₁-C₆ alkyl, C₁-C₆ alkoxy, C₃-C₆ cylcoalkyl, —CF₃, or —(CH₂)₁₋₃—CF₃;
B and C are independently selected from phenyl, pyridinyl, pyrimidinyl, furyl, thienyl or pyrrolyl groups, each optionally substituted by from 1 to 3, preferably 1 to 2, substituents selected independently from halogen, —CN, —CHO, —CF₃, —OCF₃, —OH, C₁-C₆ alkyl, C₁-C₆ alkoxy, —NH₂, —N(C₁-C₆ alkyl)₂, —NH(C₁-C₆ alkyl), —NH—C(O)—(C₁-C₆ alkyl), and —NO₂, or by a 5- or 6-membered heterocyclic or heteroaromatic ring containing 1 or 2 heteroatoms selected from O, N, and S; or
n is an integer from 0 to 3;
n₁ is an integer from 1 to 3;
n₂ is an integer from 0 to 4;
n₃ is an integer from 0 to 3;
n₄ is an integer from 0 to 2;
X₁ is selected from a chemical bond, —S—, —O—, —S(O)—, —S(O)₂—, —NH—, —C═C—,

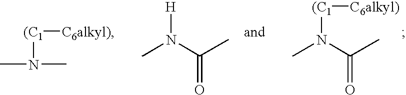

R₁ is selected from C₁-C₆ alkyl, C₁-C₆ fluorinated alkyl, C₃-C₆ cycloalkyl, tetrahydropyranyl, camphoryl, adamantyl, —CN, —N(C₁-C₆ alkyl)₂, phenyl, pyridinyl, pyrimidinyl, furyl, thienyl, napthyl, morpholinyl, triazolyl, pyrazolyl, piperidinyl, pyrrolidinyl, imidazolyl, piperizinyl, thiazolidinyl, thiomorpholinyl, tetrazolyl, indolyl, benzoxazolyl, benzofuranyl, imidazolidine-2-thionyl, 7,7-dimethyl-bicyclo[2.2.1]heptan-2-onyl, benzo[1,2,5]oxadiazolyl, 2-oxa-5-aza-bicyclo[2.2.1]heptanyl, piperazin-2-onyl and pyrrolyl groups, each optionally substituted by from 1 to 3, preferably 1 to 2, substituents independently selected from halogen, —CN, —CHO, —CF₃, —OCF₃, —OH, C₁-C₆ alkyl, C₁-C₆ alkoxy, —NH₂, —N(C₁-C₆ alkyl)₂, —NH(C₁-C₆ alkyl), —NH—C(O)—(C₁-C₆ alkyl), —NO₂, —SO₂(C₁-C₃ alkyl), —SO₂NH₂, —SO₂NH(C₁-C₃ alkyl), —SO₂N(C₁-C₃ alkyl)₂, —COOH, —CH₂—COOH, —CH₂—N(C₁-C₆ alkyl), —CH₂—N(C₁-C₆ alkyl)₂, —CH₂—NH₂, pyridinyl, 2-methyl-thiazolyl, morpholino, 1-chloro-2-methyl-propyl, C₁-C₆ thioalkyl, phenyl (further optionally substituted with one or more (e.g., 1-5, 1-4, 1-3, or 1-2) halogens), dialkylamino, —CN or —OCF₃), benzyloxy, —(C₁-C₃ alkyl)C(O)CH₃, —(C₁-C₃ alkyl)OCH₃, —C(O)NH₂, or

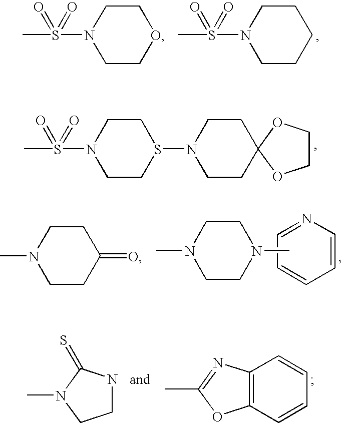

X₂ is selected from —O—, —CH₂—, —S—, —SO—, —SO₂—, —NH—, —C(O)—,

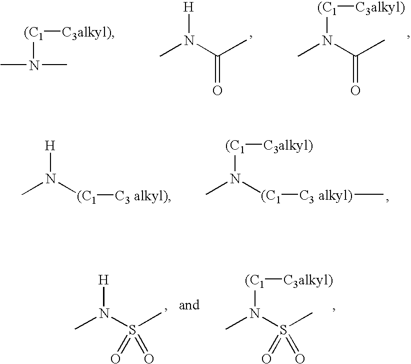

R₂ is a ring moiety selected from phenyl, pyridinyl, pyrimidinyl, furyl, thienyl and pyrrolyl groups, the ring moiety being substituted by a group of the formula —(CH₂)ₘ—CO₂H or a pharmaceutically acceptable acid mimic or mimetic; and also optionally substituted by 1 or 2 additional substituents independently selected from halogen, —CN, —CHO, —CF₃, —OCF₃, —OH, C₁-C₆ alkyl, C₁-C₆ alkoxy, C₁-C₆ thioalkyl, —NH₂, —N(C₁-C₆ alkyl)₂, —NH(C₁-C₆ alkyl), —NH—C(O)—(C₁-C₆ alkyl), and —NO₂;
R₃ is selected from H, halogen, —CN, —CHO, —CF₃, —OCF₃, —OH, C₁-C₆ alkyl, C₁-C₆ alkoxy, C₁-C₆ thioalkyl, —NH₂, —N(C₁-C₆ alkyl)₂, —NH(C₁-C₆ alkyl), —NH—C(O)—(C₁-C₆ alkyl), and —NO₂;
R₄ is selected from H, halogen, —CN, —CHO, —CF₃, —OCF₃, —OH, C₁-C₆ alkyl, C₁-C₆ alkoxy, C₁-C₆ thioalkyl, —NH₂, —N(C₁-C₆ alkyl)₂, —NH(C₁-C₆ alkyl), —NH—C(O)—(C_{r C}₆ alkyl), —NO₂, —NH—C(O)—N(C₁-C₃ alkyl)₂, —NH—C(O)—NH(C₁-C₃ alkyl), —NH—C(O)—O—(C₁-C₃ alkyl), —SO₂—C₁-C₆ alkyl, —S—C₃-C₆ cycloalkyl, —S—CH₂—C₃-C₆ cycloalkyl, —SO₂—C₃-C₆ cycloalkyl, —SO₂—CH₂—C₃-C₆ cycloalkyl, C₃-C₆ cycloalkyl, —CH₂—C₃-C₆ cycloalkyl, —O—C₃-C₆ cycloalkyl, —O—CH₂—C₃-C₆ cycloalkyl, phenyl, benzyl, benzyloxy, morpholino, pyrrolidino, piperidinyl, piperizinyl, furanyl, thienyl, imidazolyl, tetrazolyl, pyrazinyl, pyrazolonyl, pyrazolyl, oxazolyl, and isoxazolyl, the rings of each of these R₄ groups each being optionally substituted by from 1 to 3 substituents selected from the group of halogen, —CN, —CHO, —CF₃, —OH, C₁-C₆ alkyl, C₁-C₆ alkoxy, —NH₂, —N(C₁-C₆ alkyl)₂, —NH(C₁-C₆ alkyl), —NH—C(O)—(C₁-C₆ alkyl), —NO₂, —SO₂(C₁-C₃ alkyl), —SO₂NH(C₁-C₃ alkyl), —SO₂N(C₁-C₃ alkyl)₂, and —OCF₃;
each R₅ is independently H or C₁₋₃ alkyl;
R₆ is H or C₁₋₆ alkyl; and
b) a carrier or excipient system comprising:
   i) about 10 to about 50% a first solubilizer by weight of the composition;
   ii) about 5 to about 50% a second solubilizer by weight of the composition;
   iii) about 10 to about 30% a first diluent by weight of the composition; and
   iv) about 1 to about 15% a second diluent by weight of the composition.

In some embodiments, R₁ is optionally substituted phenyl; and
R is

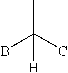

where B and C are phenyl.

In some embodiments, the present invention provides pharmaceutical compositions that include:
a) a pharmaceutically effective amount of an active pharmacological agent having Formula II:

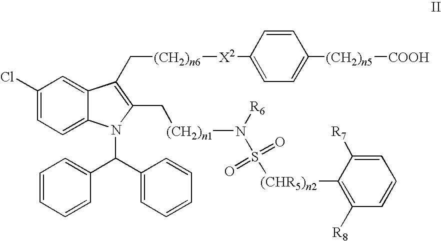

or a pharmaceutically acceptable salt thereof, wherein:
n₁ is 1 or 2;
n₂ is 1 or 2;
n₃ is 1 or 2;
n₅ is 0, 1 or 2;
X² is O, —CH₂— or SO₂;
each R₅ is independently H or C₁₋₃ alkyl;
R₆ is H or C₁₋₆ alkyl;
R₇ is selected from the group consisting of —OH, benzyloxy, —CH₃, —CF₃, —OCF₃, C₁₋₃ alkoxy, halogen, —CHO, —CO(C₁₋₃ alkyl), —CO(OC₁₋₃ alkyl), quinoline-5-yl, 3,5-dimethylisoxazol-4-yl, thiophene-3-yl, pyridin-4-yl, pyridine-3-yl, —CH₂-Q, and phenyl optionally substituted by from one to three independently selected R₃₀ groups;
R₈ is selected from the group consisting of H, —OH, —NO₂, —CF₃, —OCF₃, C₁₋₃ alkoxy, halogen, —CO(C₁₋₃ alkyl), —CO(OC₁₋₃ alkyl), quinoline-5-yl, 3,5-dimethylisoxazol-4-yl, thiophene-3-yl, —CH₂-Q, and phenyl substituted by from one to three independently selected R₃₀ groups;
Q is —OH, dialkylamino,

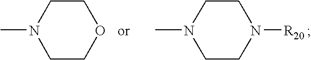

R₂₀ is selected from the group consisting of H, C₁₋₃ alkyl and —CO(C₁₋₃ alkyl); and
R₃₀ is selected from the group consisting of dialkylamino, —CN and —OCF₃;
   provided that:
i) when each R₅ is H, R₆ is H, n₅ is 0, and R₈ is H, then R₇ cannot be chlorine;
ii) when each R₅ is H, R₆ is H, n₅ is 0, X² is O or —CH₂—, and R₈ is H, then R₇ cannot be CH₃;
iii) when each R₅ is H, and R₆ is H, then R₇ and R₈ cannot both be fluorine;
iv) when each R₅ is H, R₆ is H, and X² is O, then R₇ and R₈ cannot both be chlorine;
v) when each R₅ is H, R₆ is H, X² is O, and R₈ is NO₂, then R₇ cannot be fluorine; and
vi) when each R₅ is H, R₆ is H, X² is SO₂, and R₈ is H, then R₇ cannot be fluorine or chlorine; and
b) a carrier or excipient system comprising:
   i) about 10 to about 50% a first solubilizer by weight of the composition;
   ii) about 5 to about 50% a second solubilizer by weight of the composition;
   iii) about 10 to about 30% a first diluent by weight of the composition; and
   iv) about 1 to about 15% a second diluent by weight of the composition.

In some embodiments, the compound of Formula I or Formula II has the structure of Formula III:

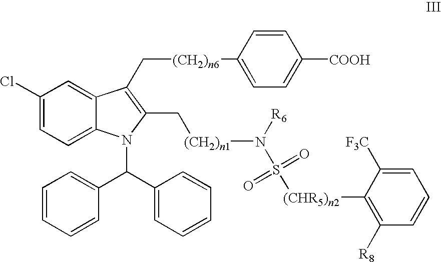

or a pharmaceutically acceptable salt thereof, wherein:
n₁ is 1 or 2;
n₂ is 1 or 2;
n₆ is 1 or 2;
R₅ is H or —CH₃;
R₆ is H or C₁₋₆ alkyl; and
R₈ is selected from the group consisting of H, —OH, —NO₂, —CF₃, —OCF₃, —OCH₃, halogen, —COCH₃, —COOCH₃, dimethylamino, diethylamino, and —CN.

In some further embodiments, the compound of Formula I or Formula II is (4-(3-{1-benzhydryl-5-chloro-2-[2-((2-trifluoromethylphenyl-methane)sulfonylamino)-ethyl]-1H-indol-3-yl}-propyl)-benzoic acid), also referred to herein as 4-(3-{5-chloro-1-(diphenylmethyl)-2-[2-({[2-(trifluoromethyl)benzyl]sulfonyl}amino)ethyl]-1H-indol-3-yl}propyl)benzoic acid, or a pharmaceutically acceptable salt thereof.

It will be understood that the C₁-C₆ fluorinated alkyl groups in the definition of R₁ may be any alkyl group of 1 to 6 carbon atoms with any amount of fluorine substitution including, but not limited to, —CF₃, alkyl chains of 1 to 6 carbon atoms terminating in a trifluoromethyl group, —CF₂CF₃, etc.

As used herein, the terms “heterocyclic” or “heterocyclyl” refer to a saturated or partially unsaturated (nonaromatic) monocyclic, bicyclic, tricyclic or other polycyclic ring system having 1-4 ring heteroatoms if monocyclic, 1-8 ring heteroatoms if bicyclic, or 1-10 ring heteroatoms if tricyclic, each of said heteroatoms being independently selected from O, N, and S (and mono and dioxides thereof, e.g., N→O⁻, S(O), SO₂. A ring heteroatom or a ring carbon can serve as the point of attachment of the heterocyclic ring to another moiety. Any atom can be substituted, e.g., by one or more substituents. Heterocyclyl groups can include, e.g. and without limitation, tetrahydropyranyl, piperidyl (piperidine), piperazinyl, morpholinyl (morpholino), thiomorpholinyl, pyrrolinyl, and pyrrolidinyl.

The term “heteroaromatic” refers to an aromatic monocyclic, bicyclic, tricyclic, or other polycyclic hydrocarbon group having 1-4 ring heteroatoms if monocyclic, 1-8 ring heteroatoms if bicyclic, or 1-10 ring heteroatoms if tricyclic, each of said heteroatoms being independently selected from O, N, and S (and mono and dioxides thereof, e.g., N→O⁻, S(O), SO₂). Any atom can be substituted, e.g., by one or more substituents. Heteroaromatic rings can include, e.g. and without limitation, pyridinyl, thiophenyl (thienyl), furyl (furanyl), imidazolyl, indolyl, isoquinolyl, quinolyl and pyrrolyl.

Pharmaceutically acceptable acid mimics or mimetics useful in the compounds of this invention include those wherein R₂ is selected from the group of:

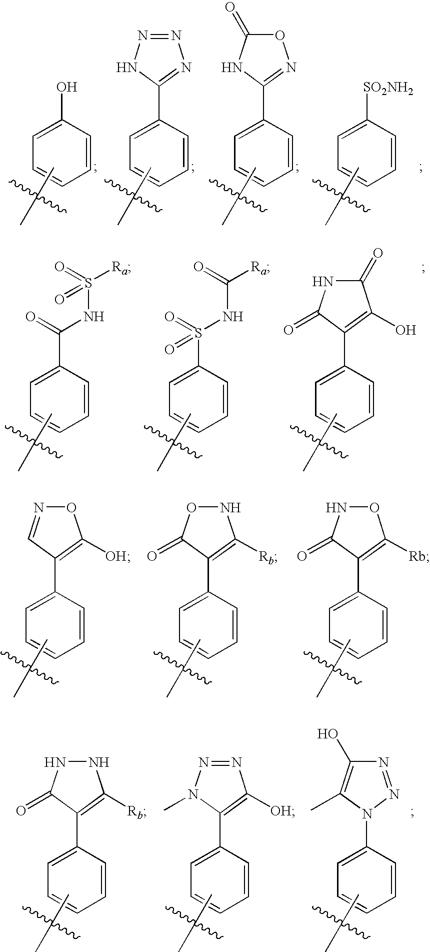

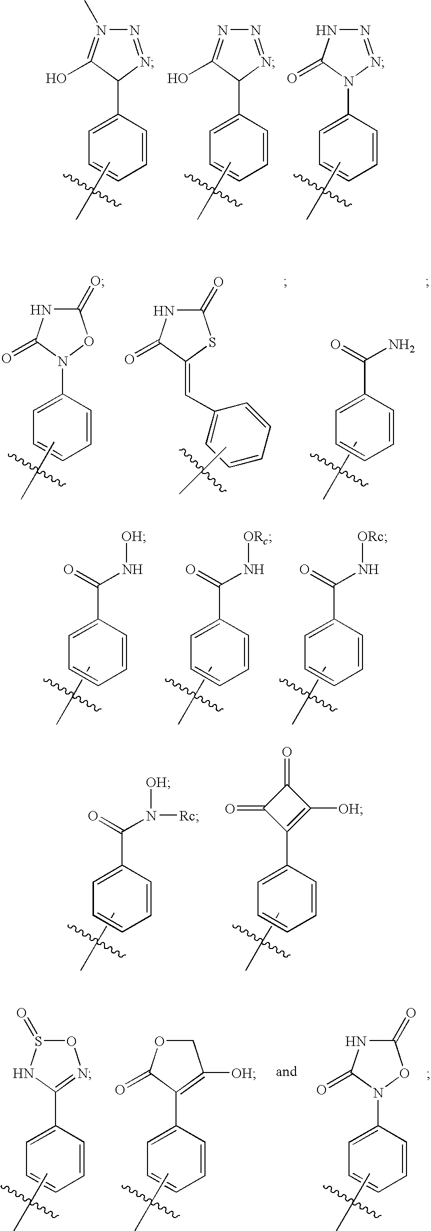

wherein Rₐ is selected from —CF₃, —CH₃, phenyl, and benzyl, with the phenyl or benzyl groups being optionally substituted by from 1 to 3 groups selected from C₁-C₆ alkyl, C₁-C₆ alkoxy, C₁-C₆ thioalkyl, —CF₃, halogen, —OH, and —COOH; R_{b} is selected from —CF₃, —CH₃, —NH₂, phenyl, and benzyl, with the phenyl or benzyl groups being optionally substituted by from 1 to 3 groups selected from C₁-C₆ alkyl, C₁-C₆ alkoxy, C₁-C₆ thioalkyl, —CF₃, halogen, —OH, and —COOH; and R_{c} is selected from —CF₃ and C₁-C₆ alkyl.

In some embodiments, the pharmaceutical compositions of the invention are liquids at ambient temperature, i.e., about 25° C. Thus, the present invention further includes dosage forms that contain the compositions of the invention, for example capsules containing compositions of the invention.

In some embodiments, the active pharmacological agent is present in an amount of from about 0.1% to about 30% by weight of the pharmaceutical compositions. In some embodiments, the active pharmacological agent is present in an amount of from about 10% to about 25% by weight of the composition; or from about 10% to about 20% by weight of the composition; or from about 15% to about 25% by weight of the composition. In some embodiments, the active pharmacological agent is present in an amount of about 20% by weight of the composition.

In some embodiments, the invention provides unit dosage forms containing the compositions of the invention. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Thus, the unit dosage forms formulations of the present invention include any conventionally used forms, including capsules, gels, oral liquids, and the like. In some embodiments, the unit dosage form is a capsule.

As will be recognized, the unit dosage forms of the invention can provide any convenient amount of the active pharmacological agent. In some embodiments, the dosage form contains, on a weight basis, the pharmacological agent in an amount of from about 0.1 mg to about 250 mg, for example from about 0.5 mg to about 200 mg; or from about 1 mg to about 150 mg; or from about 25 mg to about 125 mg; or from about 75 mg to about 125 mg. In some embodiments, the dosage form contains about 10 mg, about 25 mg, about 50 mg, about 75 mg, or about 100 mg of pharmacological agent. In some embodiments, the dosage form is a capsule that contains about 500 mg of a composition of the invention, where the composition contains 20% by weight of the pharmacological agent.

As will be recognized, the pharmacological agent can be effective over a wide dosage range, and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

Generally, the compositions of the invention contain the active pharmacological agent dissolved in a liquid carrier or excipient system, as described herein. The liquid formulations of the invention have improved properties relating to solubility, bioavailability and the like. For example, the liquid formulations of the invention have increased solubility and bioavailability compared with, for example, crystalline forms of the compound of Formula I, or its salts. The increased bioavailability associated with liquid formulations of the invention has numerous advantages including allowing for administration of lower dosages, thereby lessening chances for adverse side effects and reducing subject variability.

As described above, the pharmaceutical compositions of the invention include a carrier or excipient system that includes a first solubilizer, a second solubilizer, a first diluent and a second diluent. Both the first solubilizer and the second solubilizer can be any of a wide variety of solubilizers surfactants for liquid carriers or excipient systems known in the art, or combinations thereof. Suitable solubilizers include, for example, surfactants.

In some embodiments, the first solubilizer is selected from D-alpha-tocopheryl polyethylene glycol 1000 succinate (Vitamin E TPGS), polyethylene glycol 660 hydroxystearate, and mixtures thereof. In some preferred embodiments, the first solubilizer includes or consists of Vitamin E TPGS.

In some embodiments, the second solubilizer is selected from polyoxyl castor oils, for example polyoxyl 35 castor oil; polyoxyl hydrogenated castor oils, for example polyoxyl 40 hydrogenated castor oil; polysorbates, for example polysorbate 80, and mixtures thereof. In some embodiments, the second solubilizer comprises or consists of polyoxyl 35 castor oil. In some embodiments, the first solubilizer comprises or consists of Vitamin E TPGS and the second solubilizer comprises or consists of polyoxyl 35 castor oil.

Generally, the first solubilizer is present in an amount of from about 10% to about 50% by weight of the pharmaceutical composition. In some embodiments, the first solubilizer is present in an amount of from about 30% to about 50% by weight of the pharmaceutical composition. In some embodiments, the first solubilizer is present in an amount of from about 40% to about 50% by weight of the pharmaceutical composition. In some embodiments, the first solubilizer is present in an amount of about 45% by weight of the pharmaceutical composition.

The second solubilizer is generally present in an amount of from about 5% to about 50% by weight of the pharmaceutical composition. In some embodiments, the second solubilizer is present in an amount of from about 10% to about 30% by weight of the pharmaceutical composition. In some embodiments, the second solubilizer is present in an amount of from about 10% to about 15% by weight of the pharmaceutical composition; or in an amount of from about 5% to about 15% by weight of the pharmaceutical composition. In some embodiments, the second solubilizer is present in an amount of about 10% by weight of the pharmaceutical composition.

Both the first diluent and the second diluent can be any of a wide variety of diluents and/or solvents known in the art to be useful in liquid carriers or excipient systems, or combinations thereof. In some embodiments, the first diluent is selected from Captex® 355 (Abitec Corporation), a caprylocaproyl polyoxyglyceride, a medium chain monoglyceride, a medium chain diglyceride, a medium chain triglyceride, a triglyceride of caprylic acid, a triglyceride of capric acid, a polyethylene glycol, propylene glycol, propylene carbonate, and mixtures thereof. In some embodiments, the first diluent comprises or consists of a medium chain triglyceride, such as that sold by Abitec Corporation under the name Captex® 355. In some embodiments, the second diluent is selected from propylene carbonate, ethanol, propylene glycol, polyethylene glycol 200, polyethylene glycol 400, triacetin, and mixtures thereof. In some embodiments, the second diluent includes or consists of propylene carbonate. In some preferred embodiments, the first diluent comprises or consists of Captex® 355, and the second diluent comprises or consists of propylene carbonate.

Generally, the first diluent is present in an amount of about 10% to about 30% by weight of the pharmaceutical composition. In some embodiments, the first diluent is present in an amount of about 10% to about 25% by weight of the pharmaceutical composition; or from about 10% to about 20% by weight of the pharmaceutical composition. In some embodiments, the first diluent is present in an amount of about 15% by weight of the pharmaceutical composition.

The second diluent is generally present in an amount of about 1% to about 15% by weight of the pharmaceutical composition. In some embodiments, the second diluent is present in an amount of 1% to about 10% by weight of the pharmaceutical composition; or from about 3% to about 10% by weight of the pharmaceutical composition; or from about 6% to about 10% by weight of the pharmaceutical composition; or from about 5% to about 15% by weight of the pharmaceutical composition. In some embodiments, the second diluent is present in an amount of about 10% by weight of the pharmaceutical composition.

It will be understood that the weight percentages set forth for the first solubilizer, the second solubilizer, the first diluent and the second diluent of the compositions disclosed herein are the weight percentages that each component will comprise of a final pharmaceutical composition, including the active pharmacological agent, but without reference to a unit dosage form, or any surface covering, such as a capsule.

As will be appreciated, some components of the compositions of the invention can possess multiple functions. For example, a given component can act as both a solubilizer and a diluent. In some such cases, the function of a given component can be considered singular, even though its properties may allow multiple functionality.

In some preferred embodiments, the first solubilizer comprises or consists of Vitamin E TPGS; the second solubilizer comprises or consists of polyoxyl 35 castor oil; the first diluent comprises or consists of Captex® 355; and the second diluent comprises or consists of propylene carbonate. In some such embodiments, the Vitamin E TPGS is present in an amount of from about 10 to about 50% by weight of the composition; the polyoxyl 35 castor oil is present in an amount of from about 10 to about 50% by weight of the composition; the Captex® 355 is present in an amount of from about 10 to about 30% by weight of the composition; and the propylene carbonate in an amount of from about 1 to about 10% by weight of the composition.

In some further embodiments, the first solubilizer comprises or consists of Vitamin E TPGS in an amount of from about 40% to about 50% by weight of the composition; the second solubilizer comprises or consists of polyoxyl 35 castor oil in an amount of from about 5% to about 15% by weight of the composition; the first diluent comprises or consists of Captex® 355 in an amount of from about 10% to about 20% by weight of the composition; the second diluent comprises or consists of propylene carbonate in an amount of from about 5% to about 15% by weight of the composition; and the active pharmacological agent is present in an amount of from about 15% to about 25% by weight of the composition.

The present invention further provides processes for preparing a pharmaceutical composition that includes:
a) a pharmaceutically effective amount of an active pharmacological agent having Formula I or Formula II, or a pharmaceutically acceptable salt thereof, as described herein; and
b) a carrier or excipient system comprising:
   i) about 10 to about 50% a first solubilizer by weight of the composition;
   ii) about 5 to about 50% a second solubilizer by weight of the composition;
   iii) about 10 to about 30% a first diluent by weight of the composition; and
   iv) about 1 to about 15% a second diluent by weight of the composition;
said process comprising the steps of:
(1) mixing said first solubilizer, said second solubilizer, said first diluent and said second diluent at a temperature sufficient to form a first homogenous solution thereof;
(2) adding said pharmacological agent or a pharmaceutically acceptable salt thereof to said first homogenous solution;
(3) mixing said pharmacological agent and said first homogenous solution at a temperature sufficient to dissolve said pharmacological agent and form a second homogenous solution;
(4) optionally cooling said second homogenous solution to ambient temperature; and
(5) optionally filtering said second homogenous solution to remove undissolved particles therefrom.

In some embodiments, the compound of Formula I or Formula II has the Formula III:

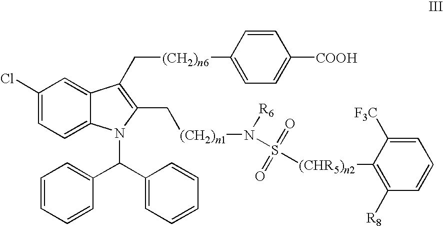

wherein:
n₁ is 1 or 2;
n₂ is 1 or 2;
n₆ is 1 or 2;
R₅ is H or —CH₃;
R₆ is H or C₁₋₆ alkyl; and
R₈ is selected from the group consisting of H, —OH, —NO₂, —CF₃, —OCF₃, —OCH₃, halogen, —COCH₃, —COOCH₃, dimethylamino, diethylamino and —CN;
   or a pharmaceutically acceptable salt thereof. In some further embodiments, the compound of Formula I is (4-(3-{1-benzhydryl-5-chloro-2-[2-((2-trifluoromethylphenyl-methane)sulfonylamino)-ethyl]-1H-indol-3-yl}-propyl)-benzoic acid), or a pharmaceutically acceptable salt thereof.

In some embodiments, the processes of the present invention further include placing at least a portion of the second homogenous solution into one or more unit dosage forms, as described herein.

Generally, it is beneficial to heat the first solubilizer, the second solubilizer, the first diluent and the second diluent while mixing, to facilitate both the mixing and dissolution of the materials. Any temperature sufficient to facilitate both the mixing and dissolution is suitable. Typically, the first solubilizer, the second solubilizer, the first diluent and the second diluent can be heated to at a temperature of about 75° C. to about 90° C. while mixing. In some embodiments, the temperature is maintained at 85+/−5° C.

Typically, the pharmacological agent is added to, and mixed with, the first solution containing the first solubilizer, the second solubilizer, the first diluent and the second diluent while the elevated temperature (e.g., from about 75° C. to about 90° C.), is maintained. In some embodiments, the temperature is maintained at 85+/−5° C. during addition of the pharmacological agent.

In some embodiments of the process of the present invention, it is advantageous to cool the second homogenous solution, for example to ambient temperature, prior to further processing, for example into unit dosage forms. In some instances, it also may be advantageous to screen the second homogenous solution to remove any undesired undissolved particles.

Generally, the second homogenous solution containing the first solubilizer, the second solubilizer, the first diluent, the second diluent and pharmacological agent is placed in unit dosage forms, as described herein. In some embodiments, the unit dosage forms are capsules.

Generally, the amount of solubilizer, diluent and pharmacological agent used will be determined by the number of unit dosage forms that is desired. As will be appreciated, the processes of the invention can be used to prepare any convenient number of unit dosage forms.

Those of skill in the art will readily recognize that simple modification of the steps outlined above, and the relative amounts of each of the components, will result in formation of a final product of desired size, strength and composition. Accordingly, the process described above can be used to make any of the pharmaceutical compositions described herein. In some embodiments, the processes are used to prepare pharmaceutical compositions where the active pharmacological agent is present in an amount of from about 0.1% to about 30% by weight of the composition; or from about 0.1% to about 20% by weight of the composition; or from about 15% to about 25% by weight of the composition; or about 20% by weight of the composition.

The present invention also provides products, including the pharmaceutical compositions and unit dosage forms, made by the processes as described herein.

As used herein, the term “a medium chain monoglyceride” refers to a monoacylglycerol having from about 8 to about 18 carbon atoms in the acyl chain.

As used herein, “a medium chain diglyceride” refers to a diacylglycerol having, independently, from about 8 to about 18 carbon atoms in each acyl chain.

As used herein, the term “a medium chain triglyceride” refers to a triacylglycerol having from about 8 to about 18 carbon atoms carbon atoms in each acyl chain. An example of medium chain triglycerides is that sold by Abitec Corporation under the name Captex® 355.

As used herein, the terms “pharmaceutically effective amount” or “therapeutically effective amount” mean the total amount of each active component of the pharmaceutical composition or method that is sufficient to show a meaningful patient benefit, i.e., treatment, healing, prevention, inhibition or amelioration of a physiological response or condition, such as an inflammatory condition or pain, or an increase in rate of treatment, healing, prevention, inhibition or amelioration of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

The term “pharmaceutically acceptable” means a non-toxic material that does not interfere with the effectiveness of the biological activity of the active ingredient(s).

Additional numerous various excipients, dosage forms, solubilizers, diluent/solvents and the like that are suitable for use in connection with the compositions of the invention are known in the art and described in, for example, Remington: The Science and Practice of Pharmacy, 20th edition, Alfonoso R. Gennaro (ed.), Lippincott Williams & Wilkins, Baltimore, Md. (2000), which is incorporated herein by reference in its entirety.

### EXAMPLES

### A. Preparation of Compounds of Formula I or Formula II

The compounds of Formula I or Formula II can be conveniently prepared from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but one skilled in the art can determine such conditions by routine optimization procedures. Those skilled in the art will recognize that the nature and order of the synthetic steps presented may be varied for the purpose of optimizing the formation of the compounds of the invention.

Preparation of compounds can involve the protection and deprotection of various chemical groups. The need for protection and deprotection, and the selection of appropriate protecting groups can be readily determined by one skilled in the art. The chemistry of protecting groups can be found, for example, in Greene, et al., Protective Groups in Organic Synthesis, 4th Ed., Wiley & Sons, 2006, which is incorporated herein by reference in its entirety.

Examples of compounds of Formula I or Formula II and methods for synthesizing them can be found in U.S. Pat. Nos. 6,797,708; 6,891,065 and 6,984,735 and U.S. patent application Ser. Nos. 10/930,534 (filed Aug. 31, 2004), 10/948,004 (filed Sep. 23, 2004), 10/989,840 (filed Nov. 16, 2004), 11/014,657 (filed Dec. 16, 2004), 11/064,241 (filed Feb. 23, 2005), 11/088,568 (filed Mar. 24, 2005), 11/140,390 (filed May 27, 2005), 11/207,072 (filed Aug. 18, 2005) and 11/442,199 (filed May 26, 2006), each of which is incorporated by reference in their entireties.

Examples of compounds of Formula I and Formula II include, but are not limited to:

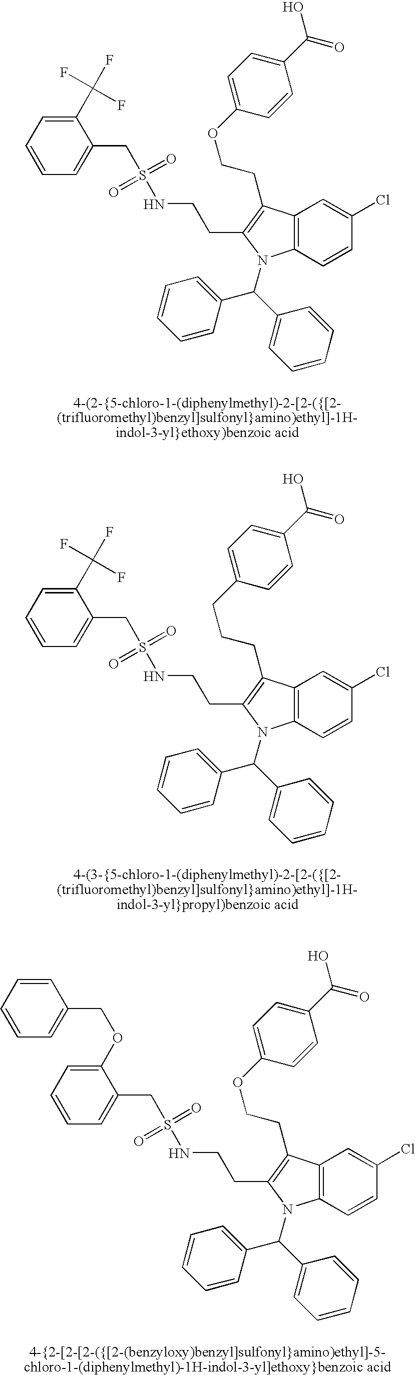

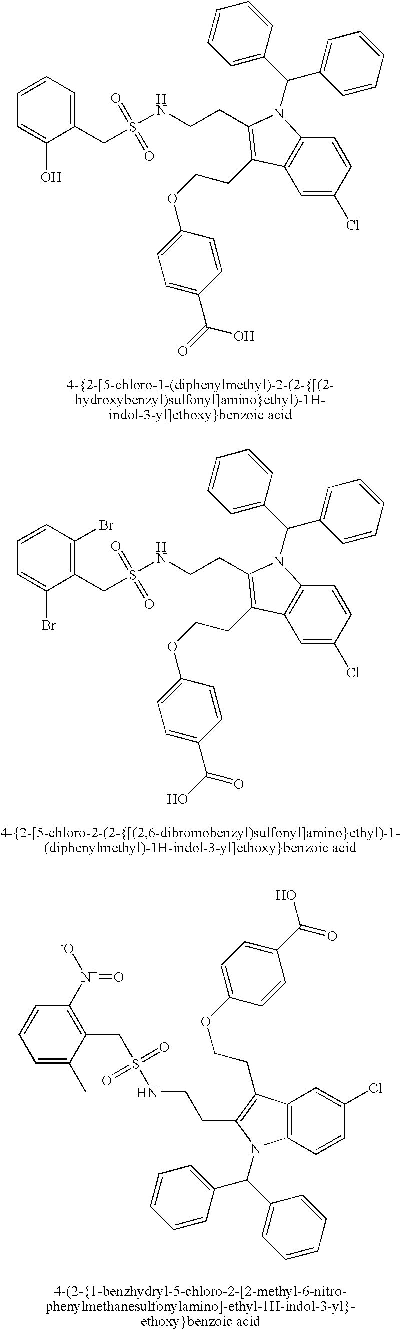

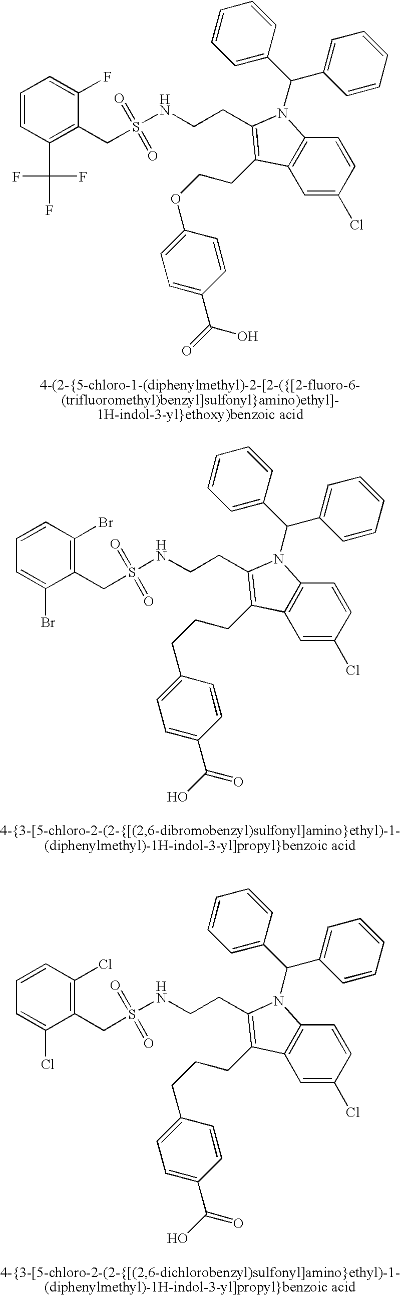

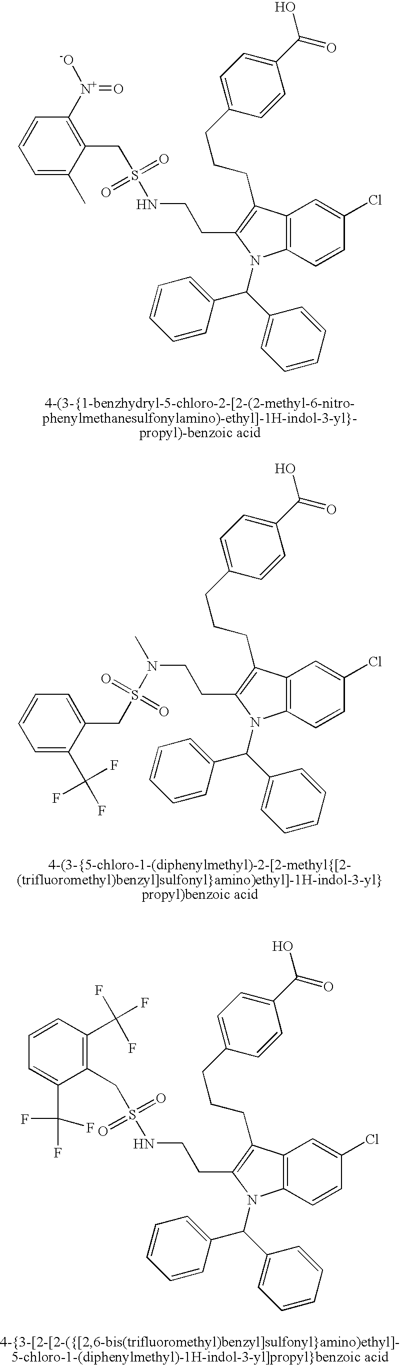

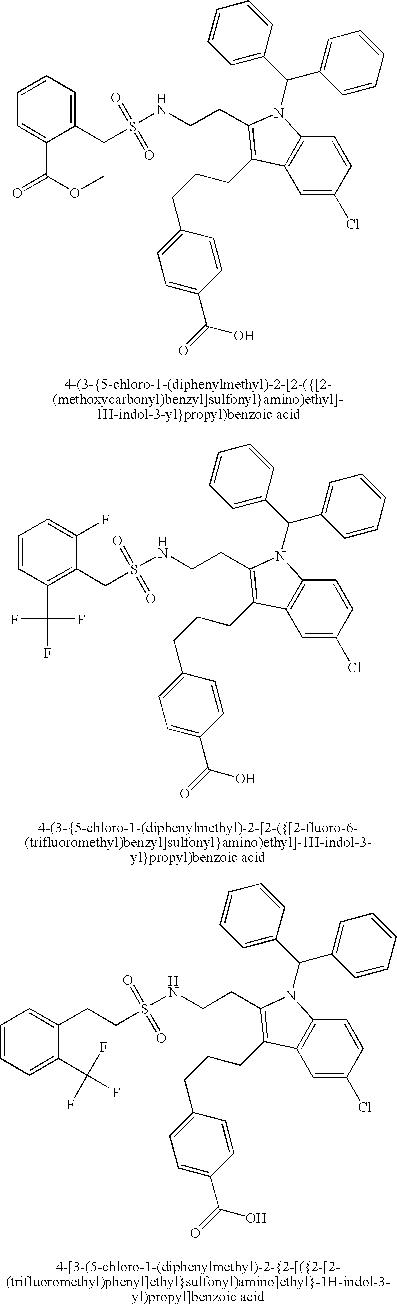

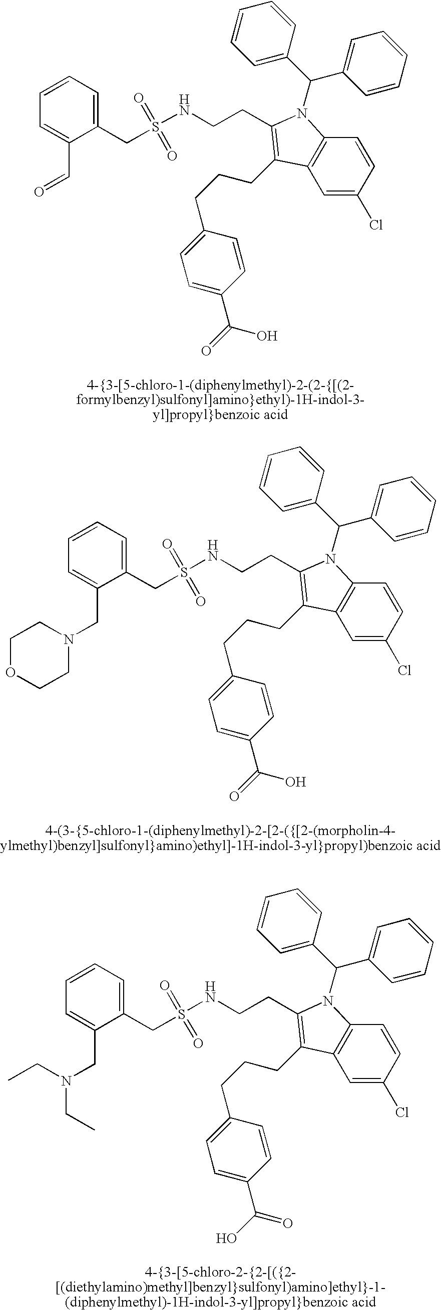

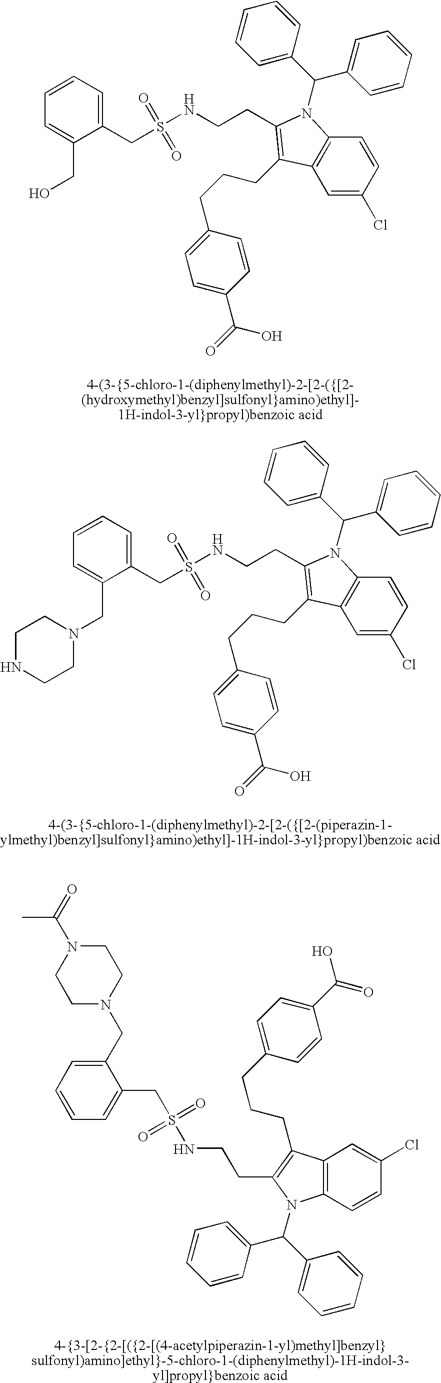

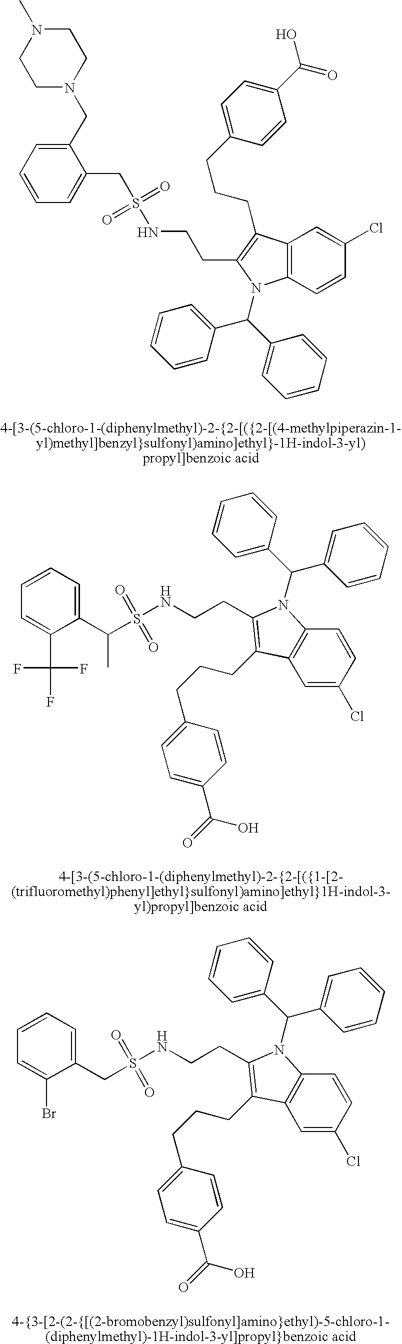

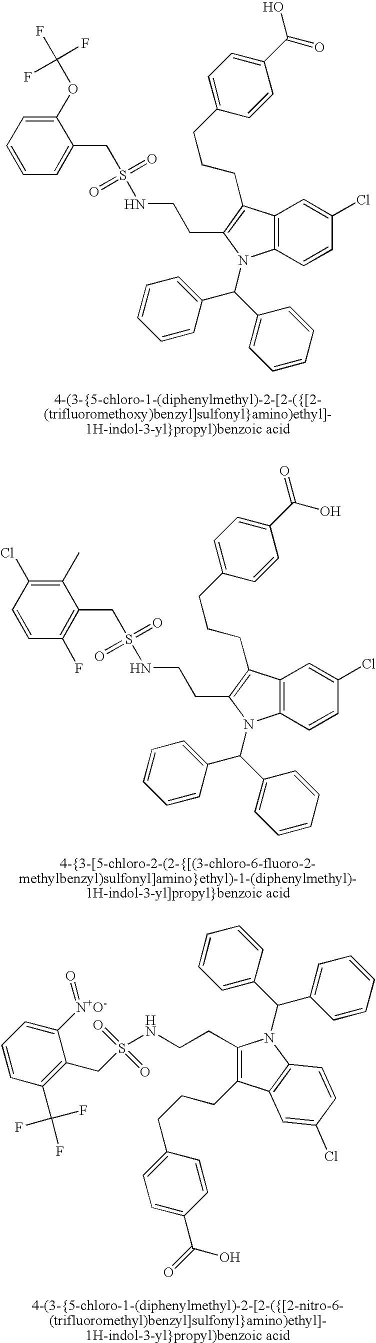

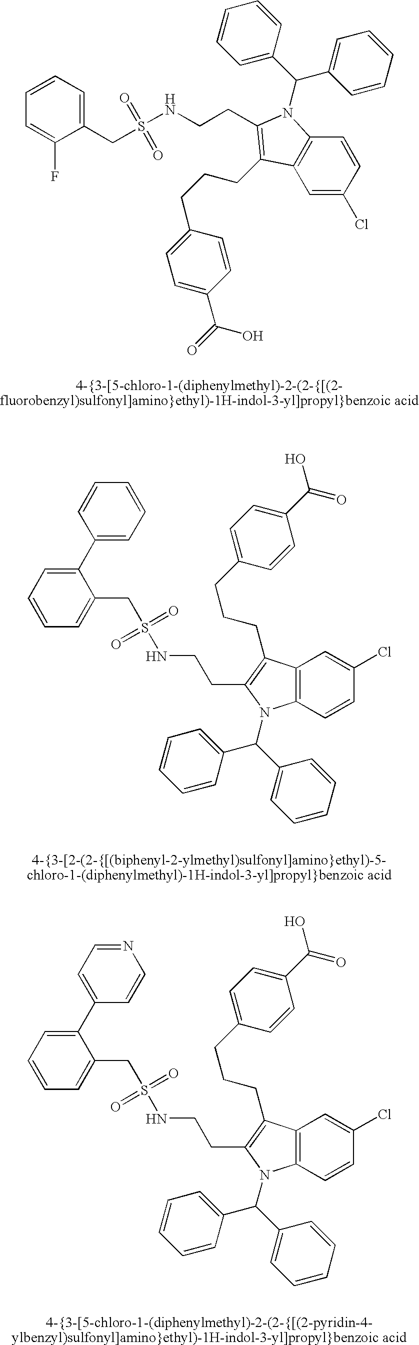

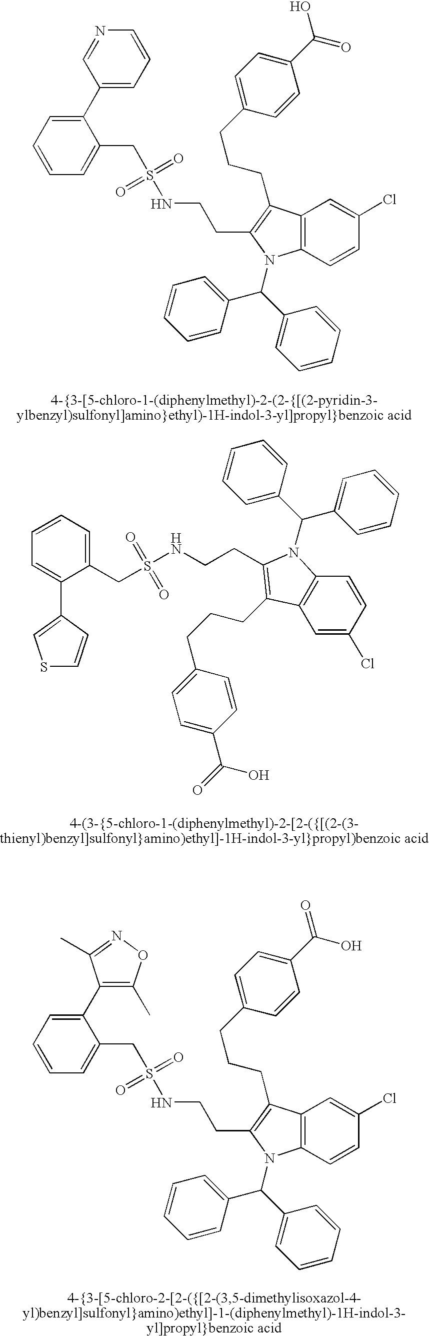

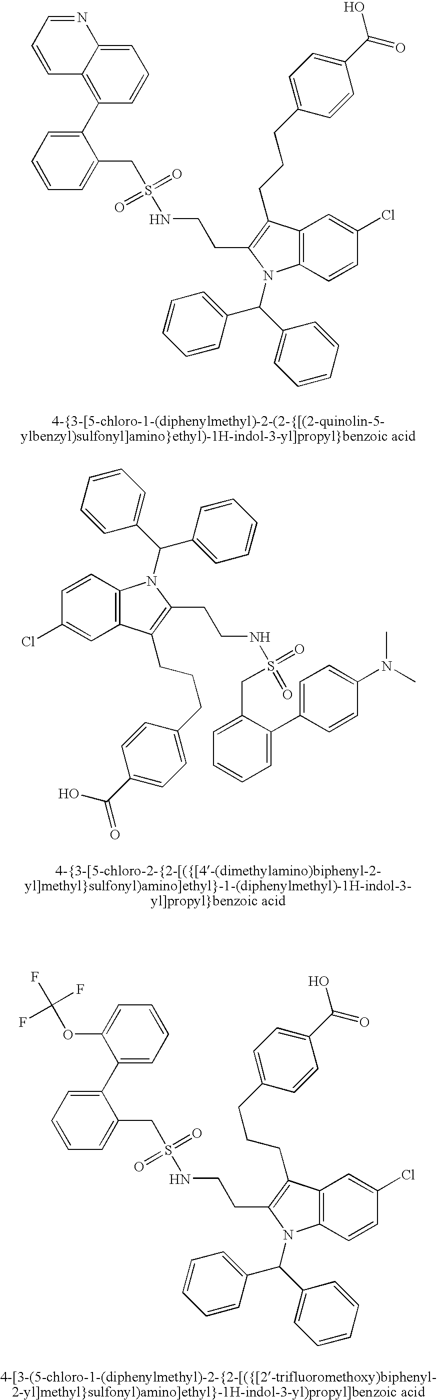

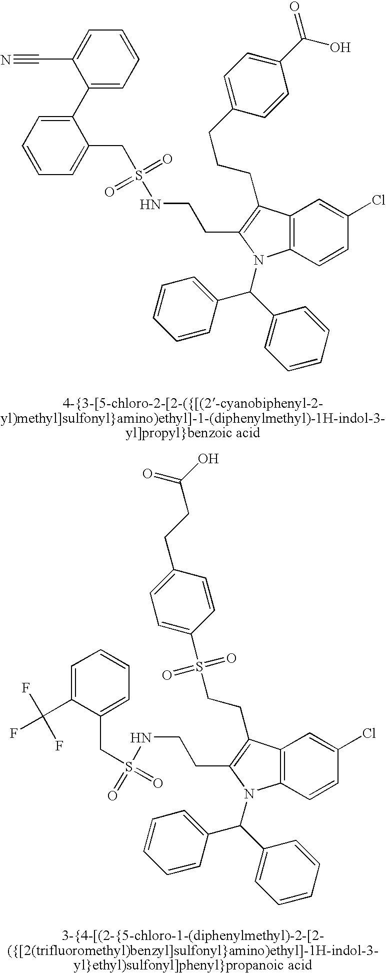

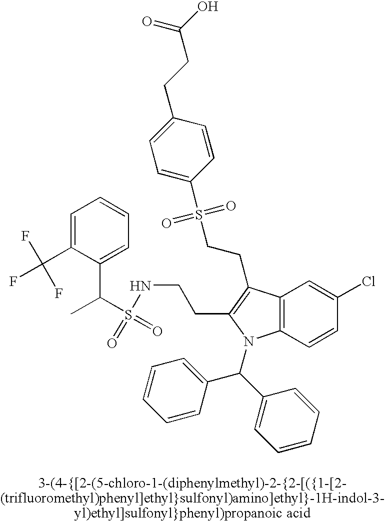

### Preparation of 100 mg Dose Capsules

A 500 mg unit dosage capsule in accordance with the invention, containing a 100 mg dose of (4-(3-{1-benzhydryl-5-chloro-2-[2-((2-trifluoromethylphenyl-methane)sulfonylamino)-ethyl]-1H-indol-3-yl}-propyl)-benzoic acid), was prepared as described in Table 1.

TABLE 1

| | | Wt % of | Weight |
|---|---|---|---|
| Component | Compound | Composition | (mg) |
| First Solubilizer | Vitamin E TPGS | 45 | 225 |
| Second Solubilizer | polyoxyl 35 castor oil | 10 | 50 |
| First Diluent | Captex ® 355 | 15 | 75 |
| Second Diluent | propylene carbonate | 10 | 50 |
| Pharmacological | (4-(3-{1-benzhydryl-5- | 20 | 100 |
| Agent | chloro-2-[2-((2- | | |
| | trifluoromethylphenyl- | | |
| | methane)sulfonylamino)- | | |
| | ethyl]-1H-indol-3-yl}- | | |
| | propyl)-benzoic acid) | | |

The pharmaceutical composition described above was prepared for administration via a capsule as follows:
1. 13.38 g of vitamin E TPGS, 3.02 g of polyoxyl 35 castor oil (Cremophor EL), 4.49 g of Captex® 355, and 3.00 g of propylene carbonate were placed into an appropriate mixing vessel equipped for temperature control.
2. The vessel was heated to 85+/−5° C. with mixing until a homogeneous solution was obtained.
3. 6.12 g of (4-(3-{1-benzhydryl-5-chloro-2-[2-((2-trifluoromethylphenyl-methane)sulfonylamino)-ethyl]-1H-indol-3-yl}-propyl)-benzoic acid), was added slowly into the solution in Step 2. The mixture was heated and mixed at 85+/−5° C. until the compound was dissolved and a homogeneous solution was obtained.
4. The resulting solution was then cooled to room temperature with mixing.
5. Size #0 capsules were then filled with 0.500 g of the finished solution from Step 4, and the capsules were sealed.

### C. Dissolution Testing

The solubility of 4-(3-{5-chloro-1-(diphenylmethyl)-2-[2-({[2-(trifluoromethyl)benzyl]sulfonyl}amino)ethyl]-1H-indol-3-yl}propyl)benzoic acid was measured at room temperature in water, acid and basic conditions. The intrinsic solubility of the free acid was below the HPLC detection limit of 31 ng/mL, whereas the anion had a solubility of 110 ng/mL.

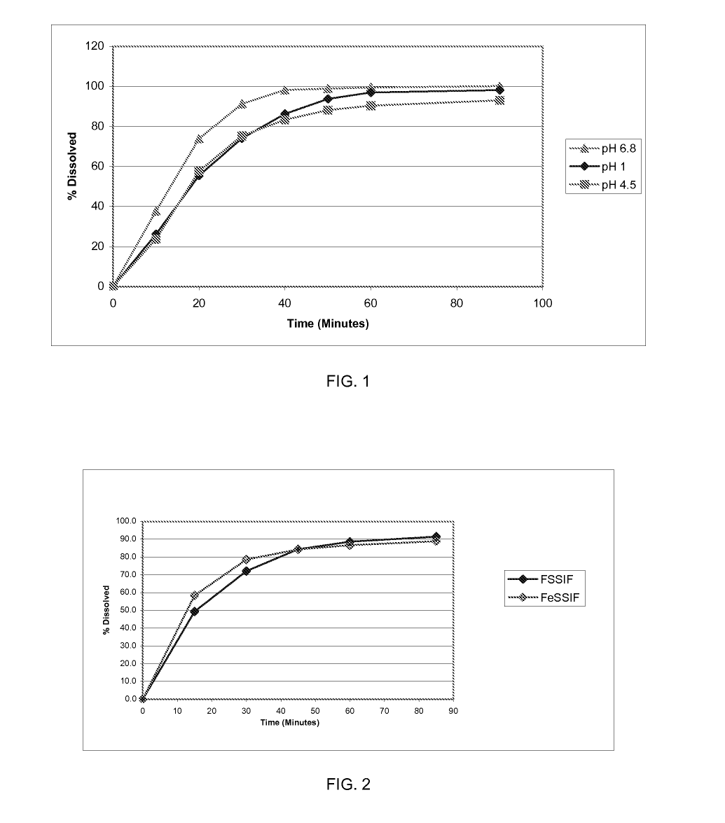

Dissolution testing was performed on 100 mg strength capsules produced according to the procedure described above. Capsules were placed in 900 mL of aqueous solutions having pH 1 (0.1 N HCl), pH 6.8 (50 mM sodium phosphate buffer) and pH 4.5 (mM sodium acetate buffer). The UV absorption of each solution was measured at various timepoints (1 mm path length, 237 nm) and the percent dissolution was calculated compared to a standard response at that wavelength. As shown in FIG. 1, the rate of dissolution was found to be similar at each pH tested.

Dissolution testing was then performed on 100 mg strength capsules produced according to the procedure described above in Fasted State Simulated Intestinal Fluid (FSSIF: 0.029 M KH₂PO₄, 5 mM sodium taurocholate, 1.5 mM lecithin, 0.22 M KCl, pH adjusted to 6.8 with NaOH) and Fed State Simulated Intestinal Fluid (FeSSIF: 0.144 M acetic acid, 15 mM sodium taurocholate, 4 mM lecithin, 0.19 M KCl, pH adjusted to 5.0 with NaOH) to simulate fed and fasted conditions in the gut. As shown in FIG. 2, there was no appreciable increase in dissolution in the simulated fed versus the fasted media.

### D. In Vivo Dog Exposure Studies

A formulation containing 4-(3-{5-chloro-1-(diphenylmethyl)-2-[2-({[2-(trifluoromethyl)benzyl]sulfonyl}amino)ethyl]-1H-indol-3-yl}propyl)benzoic acid according to the invention was studied in dogs in a high fat-fed/fasted study at approximately 12 mg/kg. To simulate the fed state, three female beagle dogs were fed a high-fat diet by oral gavage 30 minutes prior to dosing with 100 mg dose capsules as described in Table 1 above. Blood samples were drawn at 0, 0.5, 1, 2, 3, 4, 6, 8, 12 and 24 hours. The dogs were then fed ⅔ of the daily food ration after the 4 hour blood draw. Blood samples were stored on ice, centrifuged at 5° C., and the plasma was collected and stored at −70° C. The plasma samples were analyzed by LC/MS/MS to determine the amount of 4-(3-{5-chloro-1-(diphenylmethyl)-2-[2-({[2-(trifluoromethyl)benzyl]sulfonyl}amino)ethyl]-1H-indol-3-yl}propyl)benzoic acid in the sample.

To simulate the fasted state, the above procedure was repeated with the same three female beagle dogs that were fasted overnight prior to dosing, then fed after the 4 hour blood draw. The results of both the fed and fasted studies are summarized in Table 2 (reported results are the average of the data from the three test animals).

TABLE 2

| | Cₘₐₓ | AUC_{inf} | | | % Bio- | Fed/Fasted | Fed/Fasted |
|---|---|---|---|---|---|---|---|
| Formulation | (ng/mL) | (ng hr/mL) | AUC/Dose | Cₘₐₓ/Dose | availability | AUC/Dose | Cₘₐₓ/Dose |
| Fasted | 1999 | 10545 | 1127 | 213.1 | 5.93 | 3.10 | 2.31 |
| Fed | 4473 | 29844 | 3200 | 480.8 | 16.84 | | |

Data from a rat carrageenan-induced paw edema (CPE) study indicated the minimum efficacious exposure of 4-(3-{5-chloro-1-(diphenylmethyl)-2-[2-({[2-(trifluoromethyl)benzyl]sulfonyl}amino)ethyl]-1H-indol-3-yl}propyl)benzoic acid was 1360 ng*hr/ml. The data in Table 2 shows that the formulation according to the present invention results in an exposure of about 8 times the efficacious exposure in the fasted state and about 22 times the efficacious exposure in the fed state. These exposures translate into percent bioavailabilities of 5.9 and 16.8 when compared to an IV formulation (15% 4-(3-{5-chloro-1-(diphenylmethyl)-2-[2-({[2-(trifluoromethyl)benzyl]sulfonyl}amino)ethyl]-1H-indol-3-yl}propyl)benzoic acid, 10% EtOH, 75% Solutol HS-15, diluted to 2 mg/mL with sterile water for injection).

All publications mentioned herein, including but not limited to patent applications, patents, and other references, are incorporated by reference in their entirety.

The materials, methods, and examples presented herein are intended to be illustrative, and are not intended to limit the scope of the invention.

1. A pharmaceutical composition comprising
a) an excipient or carrier system comprising:
i) a first solubilizer in an amount of from about 10% to about 50% by weight of the composition;
ii) a second solubilizer in an amount of from about 5% to about 50% by weight of the composition;
iii) a first diluent in an amount of from about 10% to about 30% by weight of the composition; and
iv) a second diluent in an amount of from about 1% to about 15% by weight of the composition; and
b) a pharmaceutically effective amount of an active pharmacological agent having Formula I: 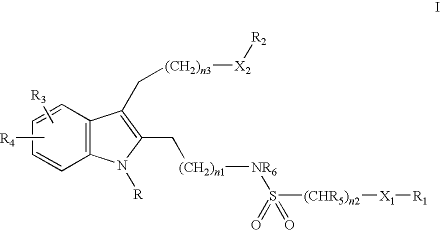
or a pharmaceutically acceptable salt thereof, wherein:
R is selected from the formulae —(CH₂)ₙ-A, —(CH₂)ₙ—S-A, and —(CH₂)ₙ—O-A, wherein A is selected from the moieties: 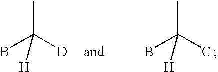
wherein
D is C₁-C₆ alkyl, C₁-C₆ alkoxy, C₃-C₆ cycloalkyl, —CF, or —(CH₂)₁₋₃—CF₃;
B and C are independently selected from phenyl, pyridinyl, pyrimidinyl, furyl, thienyl or pyrrolyl groups, each optionally substituted by from 1 to 3 substituents selected independently from halogen, —CN, —CHO, —CF₃, —OCF₃, —OH, C₁-C₆ alkyl, C₁-C₆ alkoxy, —NH₂, —N(C₁-C₆ alkyl)₂, —NH/C₁-C₆ alkyl), —NH—C(O)—(C₁-C₆ alkyl), and —NO₂, or by a 5- or 6-membered heterocyclic or heteroaromatic ring containing 1 or 2 heteroatoms selected from O, N, and S;
n is an integer from 0 to 3;
n₁ is an integer from 1 to 3;
n₂ is an integer from 0 to 4;
n₃ is an integer from 0 to 3;
n₄ is an integer from 0 to 2;
X₁ is selected from a chemical bond, —S—, —O—, —S(O)—, —S(O)₂—, —NH—, —C═C—, 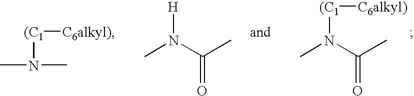
R₁ is selected from C₁-C₆ alkyl, C₁-C₆ fluorinated alkyl, C₃-C₆ cycloalkyl, tetrahydropyranyl, camphoryl, adamantyl, —CN, —N(C₁-C₆ alkyl)₂, phenyl, pyridinyl, pyrimidinyl, furyl, thienyl, naphthyl, morpholinyl, triazolyl, pyrazolyl, piperidinyl, pyrrolidinyl, imidazolyl, piperizinyl, thiazolidinyl, thiomorpholinyl, tetrazolyl, indolyl, benzoxazolyl, benzofuranyl, imidazolidine-2-thionyl, 7,7-dimethyl-bicyclo[2.2.1]heptan-2-onyl, benzo[1,2,5]oxadiazolyl, 2-oxa-5-aza-bicyclo[2.2.1]heptanyl, piperazin-2-onyl and pyrrolyl groups, each optionally substituted by from 1 to 3 substituents independently selected from halogen, —CN, —CHO, —CF₃, —OCF₃, —OH, C₁-C₆ alkyl, C₁-C₆ alkoxy, —NH₂, —N(C₁-C₆ alkyl)₂, —NH(C₁-C₆ alkyl), —NH—C(O)—(C₁-C₆ alkyl), —NO₂, —SO₂(C₁-C₃ alkyl), —SO₂NH₂, —SO₂NH(C₁-C₃ alkyl), —SO₂N(C₁-C₃ alkyl)₂, —COON, —CH₂—COOH, —CH₂—NH(C₁-C₆ alkyl), —CH₂—N(C₁-C₆ alkyl)₂, —CH₂—NH₂, pyridinyl, 2-methyl-thiazolyl, morpholino, 1-chloro-2-methyl-propyl, C₁-C₆thioalkyl, phenyl (further optionally substituted with one or more halogens, dialkylamino, —CN, or —OCF₃), benzyloxy, —(C₁-C₃ alkyl)C(O)CH₃, —(C₁-C₃ alkyl)OCH₃, —C(O)NH₂, or 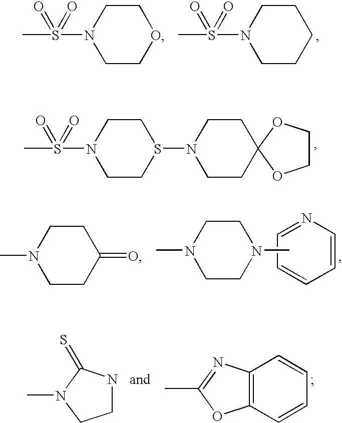
X₂ is selected from —O—, —CH₂—, —S—, —SO—, —SO₂—, —NH—, —C(O)—, 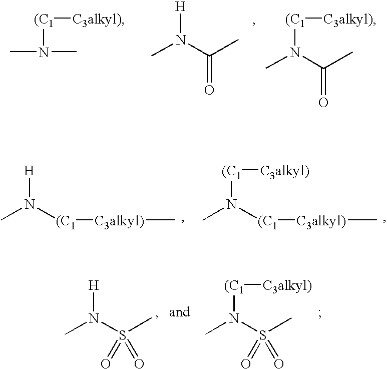
R₂ is a ring moiety selected from phenyl, pyridinyl, pyrimidinyl, furyl, thienyl and pyrrolyl groups, the ring moiety being substituted by a group of the formula —(CH₂)ₙ₄—CO₂H or a pharmaceutically acceptable acid mimic or mimetic; and also optionally substituted by 1 or 2 additional substituents independently selected from halogen, —CN, —CHO, —CF₃, —OCF₃, —OH, C₁-C₆ alkyl, C₁-C₆ alkoxy, C₁-C₆ thioalkyl, —NH₂, —N(C₁-C₆ alkyl)₂, —NH(C₁-C₆ alkyl), —NH—C(O)—(C₁-C₆ alkyl), and —NO₂;
R₃ is selected from H, halogen, —CN, —CHO, —CF₃, —OCF₃, —OH, C₁-C₆ alkyl, C₁-C₆ alkoxy, C₁-C₆ thioalkyl, —NH₂, —N(C₁-C₆ alkyl)₂, —NH(C₁-C₆ alkyl), —NH—C(O)—(C₁-C₆ alkyl), and —NO₂;
R₄ is selected from H, halogen, —CN, —CHO, —CF₃, —OCF₃, —OH, C₁-C₆ alkyl, C₁-C₆ alkoxy, C₁-C₆ thioalkyl, —NH₂, —N(C₁-C₆ alkyl)₂, —NH(C₁-C₆ alkyl), —NH—C(O)—(C₁-C₆ alkyl), —NO₂, —NH—C(O)—N(C₁-C₃ alkyl)₂, —NH—C(O)—NH(C₁-C₃ alkyl), —NH—C(O)—O—(C₁-C₃ alkyl), —SO₂—C₁-C₆ alkyl, —S—C₃-C₆ cycloalkyl, —S—CH₂—C₃-C₆ cycloalkyl, —SO₂—C₃-C₆ cycloalkyl, —SO₂—CH₂—C₃-C₆ cycloalkyl, C₃-C₆ cycloalkyl, —CH₂—C₃-C₆ cycloalkyl, —O—C₃-C₆ cycloalkyl, —O—CH₂—C₃-C₆ cycloalkyl, phenyl, benzyl, benzyloxy, morpholino, pyrrolidino, piperidinyl, piperizinyl, furanyl, thienyl, imidazolyl, tetrazolyl, pyrazinyl, pyrazolonyl, pyrazolyl, oxazolyl, and isoxazolyl, the rings of each of these R₄ groups each being optionally substituted by from 1 to 3 substituents selected from the group of halogen, —CN, —CHO, —CF₃, —OH, C₁-C₆ alkyl, C₁-C₆ alkoxy, —NH₂, —N(C₁-C₆ alkyl)₂, —NH(C₁-C₆ alkyl), —NH—C(O)—(C₁-C₆ alkyl), —NO₂, —SO₂(C₁-C₃ alkyl), —SO₂NH(C₁-C₃ alkyl), —SO₂N(C₁-C₃ alkyl)₂, and —OCF₃;
each R₅ is independently H or C₁₋₃ alkyl; and
R₆ is H or C₁₋₆ alkyl.

2. The pharmaceutical composition of claim 1, wherein
R₁ is optionally substituted phenyl; and
R is 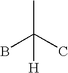
where B and C are phenyl.

3. The pharmaceutical composition of claim 1, wherein said composition is a liquid at ambient temperature.

4. The pharmaceutical composition of claim 1, wherein said active pharmacological agent is present in an amount of from about 0.1% to about 30% by weight of the composition.

5. The pharmaceutical composition of claim 1, wherein said active pharmacological agent is present in an amount of from about 10% to about 25% by weight of the composition.

6. The pharmaceutical composition of claim 1, wherein said first solubilizer is selected from the group consisting of Vitamin E TPGS, polyethylene glycol 660 hydroxystearate, and mixtures thereof.

7. The pharmaceutical composition of claim 1, wherein said first solubilizer comprises Vitamin E TPGS.

8. The pharmaceutical composition of claim 1, wherein said second solubilizer is selected from the group consisting of polyoxyl castor oils, polyoxyl hydrogenated castor oils, polysorbates, and mixtures thereof.

9. The pharmaceutical composition of claim 1, wherein said second solubilizer is selected from the group consisting of polyoxyl 35 castor oil, polyoxyl 40 hydrogenated castor oil, polysorbate 80, and mixtures thereof.

10. The pharmaceutical composition of claim 1, wherein said second solubilizer comprises polyoxyl 35 castor oil.

11. The pharmaceutical composition of claim 1, wherein said first diluent is selected from the group consisting of Captex® 355, a caprylocaproyl polyoxyglyceride, a medium chain monoglyceride, a medium chain diglyceride, a medium chain triglyceride, a triglyceride of caprylic acid, a triglyceride of capric acid, a polyethylene glycol, propylene glycol, propylene carbonate, and mixtures thereof.

12. The pharmaceutical composition of claim 1, wherein said first diluent comprises Captex® 355.

13. The pharmaceutical composition of claim 1, wherein said second diluent is selected from the group consisting of propylene carbonate, ethanol, propylene glycol, polyethylene glycol 200, polyethylene glycol 400, triacetin, and mixtures thereof.

14. The pharmaceutical composition of claim 1, wherein said second diluent comprises propylene carbonate.

15. The pharmaceutical composition of claim 1, wherein:
i) the first solubilizer is selected from the group consisting of Vitamin E TPGS, polyethylene glycol 660 hydroxystearate, and mixtures thereof;
ii) the second solubilizer is selected from the group consisting of polyoxyl castor oils, polyoxyl hydrogenated castor oils, polysorbates, and mixtures thereof;
iii) the first diluent is selected from the group consisting of Captex® 355, a caprylocaproyl polyoxyglyceride, a medium chain monoglyceride, a medium chain diglyceride, a medium chain triglyceride, a triglyceride of caprylic acid, a triglyceride of capric acid, a polyethylene glycol, propylene glycol, propylene carbonate, and mixtures thereof; and
iv) the second diluent is selected from the group consisting of propylene carbonate, ethanol, propylene glycol, polyethylene glycol 200, polyethylene glycol 400, triacetin, and mixtures thereof.

16. The pharmaceutical composition of claim 1, wherein said first solubilizer comprises Vitamin E TPGS; said second solubilizer comprises polyoxyl 35 castor oil; said first diluent comprises Captex® 355; and said second diluent comprises propylene carbonate.

17. A pharmaceutical composition comprising:
a) a carrier or excipient system comprising:
i) a first solubilizer in an amount of from about 10% to about 50% by weight of the composition;
ii) a second solubilizer in an amount of from about 5% to about 50% by weight of the composition;
iii) a first diluent in an amount of from about 10% to about 30% by weight of the composition; and
iv) a second diluent in an amount of from about 1% to about 15% by weight of the composition; and
b) a pharmaceutically effective amount of an active pharmacological agent having Formula II: 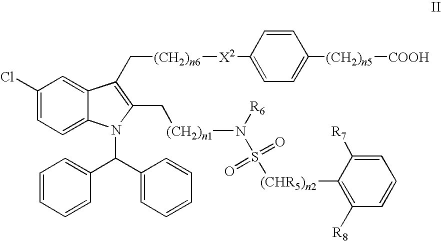
or a pharmaceutically acceptable salt thereof, wherein:
n₁ is 1 or 2;
n₂ is 1 or 2;
n₃ is 1 or 2;
n₅ is 0, 1 or 2;
X² is O, —CH₂— or SO₂;
each R₅ is independently H or C₁₋₃ alkyl;
R₆ is H or C₁₋₆ alkyl;
R₇ is selected from the group consisting of —OH, benzyloxy, —CH₃, —CF₃, —OCF₃, C₁₋₃ alkoxy, halogen, —CHO, —CO(C₁₋₃ alkyl), —CO(OC₁₋₃ alkyl), quinoline-5-yl, 3,5-dimethylisoxazol-4-yl, thiophene-3-yl, pyridine-3-yl, —CH₂-Q, and phenyl optionally substituted by from one to three independently selected R₃₀ groups;
R₈ is selected from the group consisting of H, —OH, —NO₂, —CF₃, —OCF₃, C₁₋₃ alkoxy, halogen, —CO(C₁₋₃ alkyl), —CO(OC₁₋₃ alkyl), quinoline-5-yl, 3,5-dimethylisoxazol-4-yl, thiophene-3-yl, —CH₂-Q, and phenyl substituted by from one to three independently selected R₃₀ groups;
Q is —OH, dialkylamino, 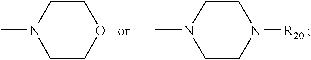
R₂₀ is selected from the group consisting of H, C₁₋₃ alkyl, and —CO(C₁₋₃ alkyl); and
R₃₀ is selected from the group consisting of dialkylamino, —CN, and —OCF₃;
provided that:
i) when each R₅ is H, R₆ is H, n₅ is 0, and R₈ is H, than R₇ cannot be chlorine;
ii) when each R₅ is H, R₆ is H, n₅ is 0, X² is O or —CH₂—, and R₈ is H, then R₇ cannot be CH₃;
iii) when each R₅ is H, and R₆ is H, then R₇ and R₈ cannot both be fluorine;
iv) when each R₅ is H, R₆ is H, and X² is O, then R₇ and R₈ cannot both be chlorine;
v) when each R₅ is H, R₆ is H, X² is O, and R₈ is NO₂, then R₇ cannot be fluorine; and
vi) when each R₅ is H, R₆ is M. X² is SO₂, and R₈ is H, then R₇ cannot be fluorine or chlorine.

18. A pharmaceutical composition of claim 17, wherein the compound of Formula II has the Formula III: 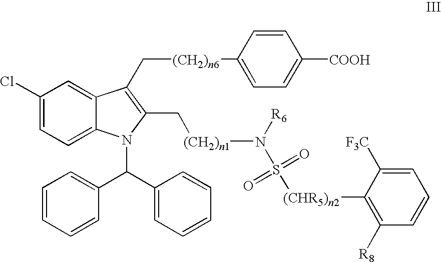
wherein:
n₁ is 1 or 2;
n₂ is 1 or 2;
n₆ is 1 or 2;
R₅ is H or —CH₃;
R₆ is H or C₁₋₆ alkyl; and
R₈ is selected from the group consisting of H, —OH, —NO₂, —CF₃, —OCF₃, —OCH₃, halogen, —COCH₃, —COOCH₃, dimethylamino, diethylamino and, —CN.

19. The pharmaceutical composition of claim 17, wherein the compound of Formula II is (4-(3-{1-benzhydryl-5-chloro-2-[2-((2-trifluoromethylphenyl-methane)sulfonylamino)-ethyl]-1H-indol-3-yl}-propyl)-benzoic acid),
or a pharmaceutically acceptable salt thereof.

20. The pharmaceutical composition of claim 17, wherein said composition is a liquid at ambient temperature.

21. The pharmaceutical composition of claim 17, wherein said active pharmacological agent is present in an amount of from about 0.1% to about 30% by weight of the composition.

22. The pharmaceutical composition of claim 17, wherein said active pharmacological agent is present in an amount of from about 10% to about 25% by weight of the composition.

23. The pharmaceutical composition of claim 17, wherein said first solubilizer is selected from the group consisting of Vitamin E TPGS, polyethylene glycol 660 hydroxystearate, and mixtures thereof.

24. The pharmaceutical composition of claim 17, wherein said first solubilizer comprises Vitamin E TPGS.

25. The pharmaceutical composition of claim 17, wherein said second solubilizer is selected from the group consisting of polyoxyl castor oils, polyoxyl hydrogenated castor oils, polysorbates, and mixtures thereof.

26. The pharmaceutical composition of claim 17, wherein said second solubilizer is selected from the group consisting of polyoxyl 35 castor oil, polyoxyl 40 hydrogenated castor oil, polysorbate 80, and mixtures thereof.

27. The pharmaceutical composition of claim 17, wherein said second solubilizer comprises polyoxyl 35 castor oil.

28. The pharmaceutical composition of claim 17, wherein said first diluent is selected from the group consisting of Captex® 355, a caprylocaproyl polyoxyglyceride, a medium chain monoglyceride, a medium chain diglyceride, a medium chain triglyceride, a triglyceride of caprylic acid, a triglyceride of capric acid, a polyethylene glycol, propylene glycol, propylene carbonate, and mixtures thereof.

29. The pharmaceutical composition of claim 17, wherein said first diluent comprises Captex® 355.

30. The pharmaceutical composition of claim 17, wherein said second diluent is selected from the group consisting of propylene carbonate, ethanol, propylene glycol, polyethylene glycol 200, polyethylene glycol 400, triacetin, and mixtures thereof.

31. The pharmaceutical composition of claim 17, wherein said second diluent comprises propylene carbonate.

32. The pharmaceutical composition of claim 17, wherein:
i) the first solubilizer is selected from the group consisting of Vitamin E TPGS, polyethylene glycol 660 hydroxystearate, and mixtures thereof;
ii) the second solubilizer is selected from the group consisting of polyoxyl castor oils, polyoxyl hydrogenated castor oils, polysorbates, and mixtures thereof;
iii) the first diluent is selected from the group consisting of Captex® 355, a caprylocaproyl polyoxyglyceride, a medium chain monoglyceride, a medium chain diglyceride, a medium chain triglyceride, a triglyceride of caprylic acid, a triglyceride of capric acid, a polyethylene glycol, propylene glycol, propylene carbonate, and mixtures thereof; and
iv) the second diluent is selected from the group consisting of propylene carbonate, ethanol, propylene glycol, polyethylene glycol 200, polyethylene glycol 400, triacetin, and mixtures thereof.

33. The pharmaceutical composition of claim 17, wherein the first solubilizer comprises Vitamin E TPGS; said second solubilizer comprises polyoxyl 35 castor oil; said first diluent comprises Captex® 355; and said second diluent comprises propylene carbonate.

34. The pharmaceutical composition of claim 17, wherein:
i) the first solubilizer comprises Vitamin E TPGS in an amount of from about 40% to about 50% by weight of the composition;
the second solubilizer comprises polyoxyl 35 castor oil in an amount of from about 5% to about 15% by weight of the composition;
iii) the first diluent comprises Captex® 355 in an amount of from about 10% to about 20% by weight of the composition;
iv) the second diluent comprises propylene carbonate in an amount of from about 5% to about 15% by weight of the composition; and
v) the active pharmacological agent is present in an amount of from about 15% to about 25% by weight of the composition.

35. A pharmaceutical dosage form comprising a composition of claim 17.

36. The pharmaceutical dosage form of claim 35, wherein the dosage form is a capsule.

37. The pharmaceutical dosage form of claim 35, wherein the active pharmacological agent is present in the dosage form in an amount of from about 0.1 mg to about 250 mg.

38. The pharmaceutical dosage form of claim 35, wherein the active pharmacological agent is present in the dosage form in an amount of from about 0.5 mg to about 200 mg.

39. The pharmaceutical dosage form of claim 35, wherein the active pharmacological agent is present in the dosage form in an amount of from about 1 mg to about 150 mg.

40. The pharmaceutical dosage form of claim 35, wherein the active pharmacological agent is present in the dosage form in an amount of from about 25 mg to about 125 mg.

41. The pharmaceutical dosage form of claim 35, wherein the active pharmacological agent is present in the dosage form in an amount of from about 75 mg to about 125 mg.

42. A process for preparing a pharmaceutical composition comprising:
a) a carrier or excipient system comprising:
i) a first solubilizer in an amount of from about 10% to about 50% by weight of the composition;
ii) a second solubilizer in an amount of from about 5% to about 50% by weight of the composition;
iii) a first diluent in an amount of from about 10% to about 30% by weight of the composition; and
iv) a second diluent in an amount of from about 1% to about 15% by weight of the composition; and
b) a pharmaceutically effective amount of an active pharmacological agent having Formula II: 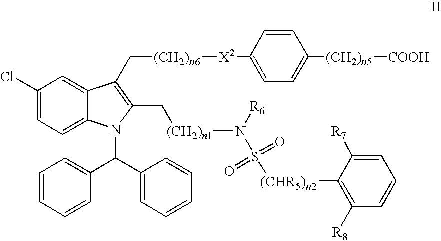
or a pharmaceutically acceptable salt thereof, wherein:
n₁ is 1 or 2;
n₂ is 1 or 2;
n₃ is 1 or 2;
n₅ is 0, 1 or 2;
X² is O, —CH₂— or SO₂;
each R₅ is independently H or C₁₋₃ alkyl;
R₆ is H or C₁₋₆ alkyl;
R₇ is selected from the group consisting of —OH, benzyloxy, —CH₃, —CF₃, —OCF₃, C₁₋₃ alkoxy, halogen, —CHO, —CO(C₁₋₃ alkyl), —CO(OC₁₋₃ alkyl), quinoline-5-yl, 3,5-dimethylisoxazol-4-yl, thiophene-3-yl, pyridin-4-yl, pyridine-3-yl, —CH₂-Q, and phenyl optionally substituted by from one to three independently selected R₃₀ groups;
R₈ is selected from the group consisting of H, —OH, —NO₂, —CF₃, —OCF₃, C₁₋₃ alkoxy, halogen, —CO(C₁₋₃ alkyl), —CO(OC₁₋₃ alkyl), quinoline-5-yl, 3,5-dimethylisoxazol-4-yl, thiophene-3-yl, —CH₂-Q, and phenyl substituted by from one to three independently selected R₃₀ groups;
Q is —OH, dialkylamino, 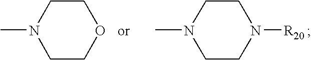
R₂₀ is selected from the group consisting of H, C₁₋₃ alkyl, and —CO(C₁₋₃ alkyl); and
R₃₀ is selected from the group consisting of dialkylamino, —CN, and —OCF₃;
provided that:
i) when each R₅ is H, R₆ is H, n₅ is 0, and H, then R₇ cannot be chlorine;
ii) when each R₅ is H, R₆ is H, n₅ is 0, X² is O or —CH₂—, and R₈ is H, then R₇ cannot be CH₃;
iii) when each R₅ is H, and R₆ is H, then R₇ and R₈ cannot both be fluorine;
iv) when each R₅ is H, R₆ is H, and X² is O, then R₇ and R₈ cannot both be chlorine;
v) when each R₅ is H, R₆ is H, X² is O, and R₈ is NO₂, then R₇ cannot be fluorine; and
vi) when each R₅ is H, R₆ is H, X² is SO₂, and R₈ is H, then R₇ cannot be fluorine or chlorine;
said process comprising:
(1) mixing the first solubilizer, second solubilizer, first diluent and second diluent to form a first homogenous solution thereof;
(2) adding the pharmacological agent or a pharmaceutically acceptable salt thereof to the first homogenous solution; and
(3) mixing the pharmacological agent and the first homogenous solution at a temperature sufficient to dissolve the pharmacological agent and form a second homogenous solution.

43. The process of claim 42, wherein step (1) further comprises heating the first solubilizer, second solubilizer, first diluent, and second diluent to a temperature sufficient to form the first homogenous solution.

44. The process of claim 43, wherein mixing the first solubilizer, second solubilizer, first diluent and second diluent is performed at a temperature of about 75° C. to about 90° C.

45. The process of claim 42, wherein the mixing of the pharmacological agent and the first homogenous solution in step (3) is performed at a temperature of about 75° C. to about 90° C.

46. The process of claim 42, further comprising the step of cooling the second homogenous solution to ambient temperature.

47. The process of claim 42, further comprising the step of filtering the second homogenous solution.

48. The process of claim 42, wherein the active pharmacological agent of Formula II has the Formula III: 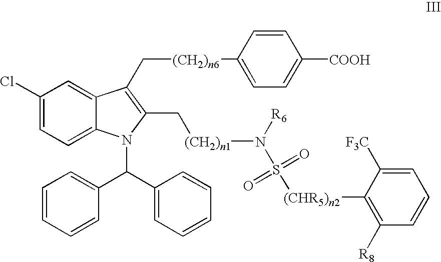
or a pharmaceutically acceptable salt thereof, wherein:
n₁ is 1 or 2;
n₂ is 1 or 2;
n₆ is 1 or 2;
R₅ is H or —CH₃;
R₆ is H or C₁₋₆ alkyl; and
R₈ is selected from the group consisting of H, —OH, —NO₂, —CF₃, —OCF₃, —OCH₃, halogen, —COCH₃, —COOCH₃, dimethylamino, diethylamino, and —CN.

49. The process of claim 42, wherein the compound of Formula II is (4-(3-{1-benzhydryl-5-chloro-2-[2-((2-trifluoromethylphenyl-methane)sulfonylamino)-ethyl]-1H-indol-3-yl}-propyl)-benzoic acid), or a pharmaceutically acceptable salt thereof.

50. The process of claim 42, further comprising placing at least a portion of the second homogenous solution into one or more unit dosage forms.

51. The process of claim 50, wherein said unit dosage form is a capsule.

52. The process of claim 42, wherein the active pharmacological agent is present in an amount of from about 0.1% to about 30% by weight of the composition.

53. The process of claim 42, wherein the first solubilizer is selected from the group consisting of Vitamin E TPGS, polyethylene glycol 660 hydroxystearate, and mixtures thereof.

54. The process of claim 42, wherein the first solubilizer comprises Vitamin E TPGS.

55. The process of claim 42, wherein the second solubilizer is selected from the group consisting of polyoxyl 35 castor oil, polyoxyl 40 hydrogenated castor oil, polysorbate 80, and mixtures thereof.

56. The process of claim 42, wherein the second solubilizer comprises polyoxyl 35 castor oil.

57. The process of claim 42, wherein the first diluent is selected from the group consisting of Captex® 355, a caprylocaproyl polyoxyglyceride, a medium chain monoglyceride, a medium chain diglyceride, a medium chain triglyceride, a triglyceride of caprylic acid, a triglyceride of capric acid, a polyethylene glycol, propylene glycol, propylene carbonate, and mixtures thereof.

58. The process of claim 42, wherein said first diluent comprises Captex® 355.

59. The process of claim 42, wherein the second diluent is selected from the group consisting of propylene carbonate, ethanol, propylene glycol, polyethylene glycol 200, polyethylene glycol 400, triacetin, and mixtures thereof.

60. The process of claim 42, wherein the second diluent comprises propylene carbonate.

61. The process of claim 42, wherein:
i) the first solubilizer is selected from the group consisting of Vitamin E TPGS, polyethylene glycol 660 hydroxystearate, and mixtures thereof;
the second solubilizer is selected from the group consisting of polyoxyl castor oils, polyoxyl hydrogenated castor oils, polysorbates, and mixtures thereof;
iii) the first diluent is selected from the group consisting of Captex® 355, a caprylocaproyl polyoxyglyceride, a medium chain monoglyceride, a medium chain diglyceride, a medium chain triglyceride, a triglyceride of caprylic acid, a triglyceride of capric acid, a polyethylene glycol, propylene glycol, propylene carbonate, and mixtures thereof; and
iv) the second diluent is selected from the group consisting of propylene carbonate, ethanol, propylene glycol, polyethylene glycol 200, polyethylene glycol 400, triacetin, and mixtures thereof.

62. The process of claim 42, wherein the first solubilizer comprises Vitamin E TPGS; the second solubilizer comprises polyoxyl 35 castor oil; the first diluent comprises Captex® 355; and the second diluent comprises propylene carbonate.

63. The process of claim 42, wherein said active pharmacological agent is present in said dosage form in an amount of from about 0.1 mg to about 250 mg.

64. The process of claim 42, wherein said active pharmacological agent is present in said dosage form in an amount of from about 0.5 mg to about 200 mg.

65. The process of claim 42, wherein the active pharmacological agent is present in said dosage form in an amount of from about 1 mg to about 150 mg.

66. The process of claim 42, wherein the active pharmacological agent is present in an amount of from about 25 mg to about 125 mg.

67. The process of claim 42, wherein the active pharmacological agent is present in an amount of from about 75 mg to about 125 mg.

68. A product made by the process of claim 42.
